(19) United States
(12) Patent Application Publication
(10) Pub. No.: US 2016/0097751 A1
(43) Pub. Date: Apr. 7, 2016

(54) APPARATUS FOR DETECTION OF PATHOLOGICAL AND NORMAL PHYSIOLOGICAL PROCESSES IN HUMANS

(71) Applicant: Evgeny Dron, Ashdod (IL)

(72) Inventor: Evgeny DRON, Ashdod (IL)

(21) Appl. No.: 14/860,725

(22) Filed: Sep. 22, 2015

Related U.S. Application Data
(60) Provisional application No. 62/059,907, filed on Oct. 5, 2014.

(51) Int. Cl. G01N 33/00

(52) U.S. Cl. CPC G01N 33/0001

(57) ABSTRACT

The present invention is an apparatus for detection of pathological and normal physiological processes in humans and it comprises an electronic nose adapted to detect volatile and non-volatile molecules produced by a human body and a computerized system connected to the electronic nose and to a remote server, and further adapted to receive a molecule detection signals from the electronic nose, and yet further adapted to compare the detected molecules to the molecular combinations stored in its memory, said molecular combinations are indicative of various pathological and/or normal physiological processes in humans.

### PRIORITY

This patent application is a non-provisional utility application that claims priority from a provisional application having Ser. No. 62/059,907 filed on Oct. 5, 2014, which is hereby incorporated by reference in its entirety.

### BACKGROUND

1. Technical Field

The present invention relates to a toilet bowl provided with a bodily fluids analysis function which is capable of sampling and analyzing bodily fluids (such as urine) excreted by individuals in a toilet bowl. More specifically, the present invention relates to a body fluid analysis apparatus which is adapted to be mounted on a standardized toilet bowl fixture commercially available on the market.

2. Related Art

In recent years the importance of health care has been receiving an increasing attention. A human body secretes various chemicals such as hormones and proteins, and excretes waste products of metabolism such as urine, feces and sweat which also contain a large number of various chemicals. These chemicals are an important source of information that represents a health condition of an individual, various dysfunctions as well as normal physiological processes can be detected advantageously in a non-invasive manner by performing quantitative analysis of certain urine constituents, such as hormones, proteins and other substances. Several known toilets are toilets having a urine analysis function. These toilets are capable of performing sampling and analysis of urine so as to assist the individuals in monitoring their health by making use of toilets provided in residences, offices and other facilities.

For instance, JP-A-59-217844 of Toto Ltd., U.S. Pat. No. 4,961,431 to Ikenaga et al., U.S. Pat. No. 4,962,550 to Ikenaga et al., U.S. Pat. No. 4,982,741 to Saito et al., U.S. Pat. No. 5,073,500 to Saito et al., propose to form a urine sampling cavity or sampling well on the bowl surface of a water closet bowl fixture to sample a quantity of urine excreted into the toilet bowl, the urine sample being subjected to urine analysis by way of liquid chromatographic process, colorimetric analysis process, or voltammetric process.

The advantage of these sampling systems which are designed to make use of the urine sampling cavity or sampling well formed on the bowl surface is that urine excreted into the toilet bowl is received and collected by a considerably wide surface area of the bowl so that urine is readily sampled regardless of the direction of urination or the variation in the trajectory of urine column. Therefore, an adequate quantity of urine necessary for urine analysis can easily be sampled even in the case of elderly people who are apt to suffer from the shortage of the amount of urine per urination.

However, the problem associated with these systems is that they require a special-purpose toilet bowl fixture provided with a urine sampling cavity on the bowl surface for the purposes of sampling of urine so that a standard type toilet bowl fixture having the conventional bowl configuration cannot be used. Accordingly, in order to reform an existing toilet equipped with the conventional standard bowl fixture into a toilet having a urine analysis function, the existing conventional bowl fixture must first be removed and then a special purpose bowl fixture envisioned for sampling and urine analysis must be installed. This involves a great deal of labor and expenses for reform works and necessitates to discard the existing bowl fixture.

Therefore, the primary object of the present invention is to provide an analysis apparatus which is capable of performing sampling and analysis of body fluids and non-fluids (urine, feces, sweat and etc.) and which can be readily mounted on a standard toilet bowl fixture and is easy to use.

### SUMMARY OF INVENTION

The present invention is an apparatus for detection of pathological and normal physiological processes in humans, comprising an electronic nose with discrete detectors, adapted to detect volatile and non-volatile molecules produced by a human body; and a computerized system connected to the electronic nose and to a remote server, and adapted to receive a molecule detection signals from the electronic nose, and further adapted to compare the detected molecules to the molecular combinations stored in its memory, said molecular combinations are indicative of various pathological and/or normal physiological processes in humans.

The apparatus of present invention is further adapted to send an electronic notification such as email and/or SMS to a predetermined address if the molecules identified by the electronic nose are identical to the molecular combinations stored in the memory of the computerized system.

The apparatus for detection of pathological and normal physiological processes in humans of present invention may be installed on a standard or smart toilet bowl or it may be integrated in a toilet bowl structure during the manufacturing process of the said toilet bowl.

### BRIEF DESCRIPTION OF THE DRAWINGS

FIG. 1—Schematically shows an apparatus for detection of pathological and normal physiological processes in humans mounted in a toilet bowl.

FIG. 2—Schematically shows the main components of an apparatus for detection of pathological and normal physiological processes in humans according to an embodiment of the present invention.

### DETAILED DESCRIPTION

In the following detailed description of the invention, numerous specific details are set forth in order to provide a thorough understanding of the invention. However, it will be obvious to one skilled in the art that the invention may be practiced without these specific details. In other instances, well known methods, procedures, components, and elements have not been described in detail so as not to unnecessarily obscure aspects of the invention.

It will be readily understood that the components of the present invention, as generally described and illustrated in the figures, may be arranged and designed in a wide variety of different configurations. Thus, the following more detailed description of the embodiments of the apparatus and methods of the present invention, as represented in the figures, is not intended to limit the scope of the invention, but is merely representative of selected embodiments of the invention.

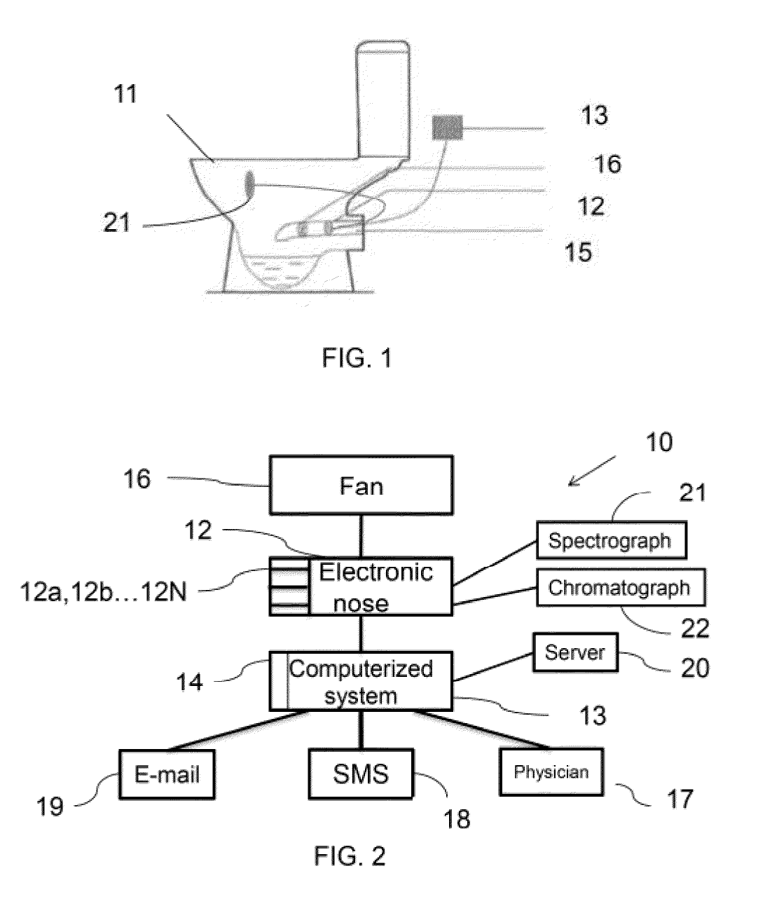

FIG. 1 and FIG. 2 depict embodiments of an apparatus 10 for detection of pathological and normal physiological processes in humans. The apparatus 10 is adapted to be mounted on or in a vicinity of a standard toilet bowl 11 and comprises an electronic nose 12 which includes a plurality of sensors 12a, 12b, 12c, . . . 12N for detection of volatile and not volatile molecules. At least one of plurality of sensors located in the electronic nose 12 is a nanoelectromechanical sensor, similar to one described in Unterreidoneier, Quirin P., Eva M. Weig, and Jörg P. Kotthaus, “Universal transduction scheme for nanomechanical systems based on dielectric forces,” Nature 458.7241 (2009): 1001-1004. Said sensor contains a plurality of resonating nano-structures coated with a molecule adsorption layer, and the resonance frequency of each structure is changed when it adsorbs one or more molecules.

The electronic nose 12 may also comprise a plurality of nano-transistor based sensors that change their electrical characteristics upon adsorption of one or more molecules from the surroundings.

The electronic nose 12 may comprise a photoionization detector for detecting volatile molecules and be connected to a discrete detectors such as a chromatograph 22 and a spectrograph 21 for detecting non-volatile molecules from a body fluid or non-fluid.

The electronic nose 12 is used as odor sensor and “smells” various volatile and non-volatile molecules produced by a human body and excreted in bodily fluids, such as urine or sweat. The electronic nose 12 is connected to a computerized system 13 that receives detection signals from the electronic nose 12 and compares the recognized molecules or their combination to a combinations stored in its memory 14 or on remote server 20. The molecule list or molecule combinations stored in the memory 14 or on the remote server 20 of the computerized system 13 are indicative of various pathological and normal physiological processes in humans. For example a detection of ketones in human urine is indicative of a diabetes. Detection of increased levels of estradiol in urine of a female human may be indicative of an incoming ovulation. Detection of a combination of fourteen specific molecules is indicative of a prostate cancer in a male human.

After the recognition of the specific molecules or the molecule combination by the computerized system 13, the computerized system 13 sends an SMS 18 or e-mail 19 notification to a predetermined address, or to a physician 17. The apparatus 10 is also adapted to recognize and identify each user either by a finger print detection or by a smart card identification. The computerized system 13 and the memory 14 may be updated from the remote server 20 by an user or automatically.

The apparatus 10 may also comprise an air pipe 15 with a fan 16 for pumping air and volatile molecules towards the electronic nose 12. In this case the chromatograph 22 and the spectrograph 21 are located outside the air pipe 15.

An electronic nose may also have a direct contact with liquids contained in the toilet bowl for detecting a non-volatile compounds produced and excreted by humans.

The apparatus 10 for detection of pathological and normal physiological processes in humans may be installed on a standard or smart toilet bowl or it may be integrated in a toilet bowl structure during the manufacturing process of the said toilet bowl.

Having thus described the present invention by reference to certain of its preferred embodiments, it is noted that the embodiments disclosed are illustrative rather than limiting in nature and that a wide range of variations, modifications, changes, and substitutions are contemplated in the foregoing disclosure and, in some instances, some features of the present invention may be employed without a corresponding use of the other features.

1. An apparatus for detection of pathological and normal physiological processes in humans, comprising:
a. an electronic nose adapted to detect volatile molecules produced by a human body and wherein said electronic nose is connected to at least one discrete detector that detects non-volatile molecules produced by a human body; and
b. a computerized system connected to the electronic nose and to a remote server, and adapted to receive a molecule detection signals from the electronic nose, and further adapted to compare the detected molecules to the molecular combinations stored in its memory, said molecular combinations are indicative of various pathological and/or normal physiological processes in humans.

2. The apparatus of claim 1, further adapted to send an electronic notification such as email and/or SMS to a predetermined address if the molecules identified by the electronic nose are identical to the molecular combinations stored in the memory of the computerized system.

3. The apparatus of claim 2, wherein said electronic nose comprises: at least one nanoelectromechanical sensor comprised of resonating nano-structures, plurality of nano-transistor based sensors that change their electrical characteristics upon adsorption of one or more molecules from the surroundings, and a photoionization detector.

4. The apparatus of claim 3, wherein said electronic nose is connected to a chromatograph and a spectrograph.

5. (canceled)

6. (canceled)

7. The apparatus of claim 4, wherein said apparatus is removably attached to a toilet bowl.

8. The apparatus of claim 4, wherein said apparatus is integrated in a toilet bowl.
